(19) United States
(12) Patent Application Publication
(10) Pub. No.: US 2008/0027300 A1
(43) Pub. Date: Jan. 31, 2008

(54) OXIMETRY PROBE ASSEMBLY HAVING A FLUID SEAL

(75) Inventors: Brian Barclay, Pleasant Prairie, WI (US); John S. Norman, Gurnee, IL (US); Anis Rahman, Kenosha, WI (US); William Rudzena, Johnsburg, IL (US); Tage Grant, Chicago, IL (US); Bradley Gould, Evanston, IL (US); Hrishikesh Choudhury, Gurnee, IL (US)

Correspondence Address: BRIAN R. WOODWORTH, 275 N. FIELD DRIVE, DEPT. NLEG BLDG H-1, LAKE FOREST, IL, 60045-2579, US

(73) Assignee: HOSPIRA, INC., Lake Forest, IL (US)

(21) Appl. No.: 11/830,998

(22) Filed: Jul. 31, 2007

Related U.S. Application Data
(60) Provisional application No. 60/834,496, filed on Jul. 31, 2006.

(51) Int. Cl. A61B 5/1459

(52) U.S. Cl. 600/339

(57) ABSTRACT

An oximetry probe assembly includes fluid seal assembly taking the form of a manually actuated plunger which moves relative to a barrel having a seal placed therein. Depression of the plunger causes the seal to be deformed so as to permit insertion of an oximetry probe through the seal and advancement towards a central venous catheter. When the plunger is released, the seal is compressed against the barrel and the sheath of the oximetry probe to thereby form a fluid-tight seal. The plunger and barrel are preferably configured as a one-handed grasping assembly whereby the user may hold the plunger and barrel with one hand, press the plunger with the thumb, and advance the oximetry probe through the fluid seal assembly with the other hand.

### CROSS-REFERENCE TO RELATED APPLICATIONS

This application claims priority of U.S. Provisional Patent Application Ser. No. 60/834,496 filed on Jul. 31, 2006, which is expressly incorporated herein by reference and made a part hereof.

### BACKGROUND

A. Field

This disclosure relates to the field of oximetry probes and more particularly to an oximetry probe assembly having a fluid seal arrangement for preventing fluid therethrough.

B. Related Art

Oximetry probes are devices used for measuring the level of oxygen saturation of the blood of a patient, e.g., when the patient is in an intensive care setting. Such probes typically have a first optical fiber configured to carry afferent light from a light source to the patient's blood and a second optical fiber configured to collect the efferent light reflected from the patient's blood and to carry the efferent light to a system that analyzes the afferent and efferent light in order to determine the level of oxygen contained in the patient's blood. The optical fibers are typically positioned within a lumen of a catheter configured for insertion into the patient's blood stream. In some cases the catheter defines a separate guidewire lumen configured to receive a guidewire, thereby enabling the catheter to be delivered to a target location within the patient's bloodstream by sliding the catheter over a pre-positioned guidewire.

A representative prior art oximetry probe is described by Rivers, U.S. Pat. No. 5,673,694, the content of which is incorporated by reference herein. Rivers provides a detailed discussion of the use of oximetry probes for measuring central venous oxygenation saturation.

### SUMMARY OF THE INVENTION

In a first aspect of the present invention, an oximetry probe assembly is provided. The oximetry probe assembly includes an oximetry probe (e.g., a central venous oximetry probe) comprising an elongate housing defining a lumen and having one or more optical fibers positioned within the lumen, the optical fibers being constructed for transmitting afferent and efferent optical signals in known fashion. The assembly further includes a body member or barrel defining an interior lumen constructed to receive the oximetry probe therethrough. A fluid seal assembly is positioned within the lumen of the body member. The fluid seal assembly includes a seal member that substantially fluidly seals the lumen of the body member. The seal member is pre-slit such that the oximetry probe can be inserted therethrough. The seal and the pre-slit are formed to provide a fluid-tight seal about an oximetry probe positioned therethrough.

A tube is provided for receiving the oximetry probe. The tube has a first end connected to the outlet port of the fluid seal assembly and a second end connected to a stop member. The stop member limits the insertion distance of the oximetry probe into the tube, thereby correctly positioning the tip of the oximetry probe relative to the central venous catheter for making measurements of blood oximetry.

The fluid seal assembly further includes a movable plunger constructed and positioned to deform the seal when the movable plunger is in an activated position. Deformation of the seal causes the slit open, thereby facilitating manual insertion of the oximetry probe through the fluid seal assembly.

In one embodiment, the fluid seal assembly further includes a barrel having finger grips, the barrel being configured to receive the plunger. The finger grips on the barrel, and a handle portion of the plunger, together form a one-handed grasping assembly, whereby a user may grasp the plunger and finger grips with one hand and depress the plunger (e.g., with the thumb) relative to the barrel to thereby move the plunger into its activated position, deform the seal, and permit the oximetry probe to be advanced through the fluid seal assembly. While the user is depressing the plunger with a first hand, the oximetry probe can be advanced through the fluid seal assembly with user's other hand until the distal end of the oximetry probe contacts the stop member, thereby properly positioning the oximetry probe. The user then releases the plunger. The seal then forms a fluid-tight seal about the oximetry probe.

In a second aspect, a fluid seal assembly is provided for an oximetry probe in the form of an elongate tubular body. The fluid seal assembly includes a plunger comprising a body having a first portion having an inlet port for receiving the oximetry probe and a second portion forming a channel. The assembly further includes a barrel having a tubular body with an outlet port. The plunger and barrel are adapted for insertion of the oximetry probe through the fluid seal assembly from the inlet port of the plunger, through the channel and barrel to the outlet port. The assembly further includes a resilient, deformable seal placed within the barrel. The seal forms a fluid seal about the oximetry probe when the oximetry probe is inserted through the fluid seal assembly. The plunger and barrel are adapted for relative movement therebetween. Movement of the plunger towards the barrel actuates (i.e., deforms) the seal. Deformation of the seal facilitates insertion and advancement of the oximetry probe through the fluid seal assembly. When the plunger is released, the seal returns to its unstressed form to create a fluid seal about the probe.

Several alternative configurations of the seal are contemplated. In one embodiment, the seal includes an elongate cylindrical portion having a longitudinal axis and an integral body portion. An opening feature in the form of a slit or a channel is formed in the body portion in substantial alignment with the longitudinal axis. During activation by the plunger, the slit opens to facilitate insertion of the oximetry probe through the seal. In one embodiment, the seal further includes a lip seal opposite from the cylindrical portion. The lip seal forms a seal about the oximetry probe's sheath to create a second fluid seal. In one embodiment, the elongate cylindrical portion has a bellows construction.

In yet another aspect of this disclosure, a method is provided of advancing an oximetry probe having a distal end through a fluid seal assembly. The method includes the steps of:

inserting the distal end of the oximetry probe into a fluid seal assembly comprising a plunger, a barrel and a seal disposed within the barrel, the plunger having an inlet port for receiving the oximetry probe, the barrel having an outlet port;

(b) further inserting the oximetry probe through the fluid seal assembly until the distal end of the oximetry probe is proximate to the seal;

manually moving the plunger relative to the barrel to thereby deform the seal with the plunger and facilitate advancement of the oximetry probe through the seal; and

(d) while the plunger is in its activated position, further advancing the oximetry probe through the fluid seal assembly whereby the distal end of the oximetry probe may be advanced through the outlet port of the barrel.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is an illustration of an oximetry probe assembly in accordance with one embodiment of this disclosure.

FIG. 2 is a perspective, cross-sectional view of the fluid seal assembly of Figure

FIG. 3 is a perspective view of the plunger component of the fluid seal assembly of FIGS. 1 and 2 with the cover removed.

FIG. 4 is a further perspective view of the plunger of FIGS. 1 and 2 with the cover removed.

FIG. 5 is a cross-sectional view of the plunger of FIGS. 1 and 2.

FIG. 6 is a perspective view of one embodiment of a cover that fits over the plunger of FIGS. 3 and 4 as seen from above.

FIG. 7 is a further perspective view of the cover of FIG. 6 as seen from below.

FIG. 8 is a perspective view of a tubular barrel component of the fluid seal assembly of FIGS. 1 and 2.

FIG. 9 is a perspective view of a cover which is placed over the finger grips of the barrel of FIG. 8.

FIG. 10 is a further perspective view of the tubular barrel of FIGS. 1, 2 and 8.

FIG. 11 is a cross-sectional view of the barrel of FIGS. 8 and 10, with the seal piece not shown for purposes of clarity.

FIG. 12 is a cross-sectional view of the barrel of FIGS. 8, 10 and 11 but also showing the seal piece placed therein.

FIG. 13 is a further cross-sectional view of the barrel of FIGS. 8 and 10, with the seal piece not shown for purposes of clarity. FIG. 13A is a detail of a portion of the barrel.

FIG. 14 is a cross-sectional view of the plunger and barrel and seal piece with the plunger biased to its inactivated position by a biasing spring.

FIG. 15 is a side view of one embodiment of a seal for the fluid seal assembly of FIGS. 1, 2 and 12. FIG. 15A is a cross-sectional view of the seal of FIG. 15 along the lines 15A-15A. FIG. 15B is a detail view of the lip seal portion of the seal. FIG. 15C is a view of a slit which is formed in the seal; the slit is opened to permit advancement of the oximetry probe by actuation of the plunger of FIGS. 1, 2 and 14.

FIG. 16 is a side view of a second embodiment of a seal for the fluid seal assembly of FIGS. 1, 2 and 12, as an alternative to that of FIG. 15. FIG. 16A is a cross-sectional view of the seal of FIG. 16 along the lines 16A-16A.

FIG. 17 is a side view of a third embodiment of a seal having a bellows configuration for the fluid seal assembly of FIGS. 1, 2 and 12, as an alternative to that of FIGS. 15 and 16. FIG. 17A is a cross-sectional view of the seal of FIG. 17 along the lines 17A-17A. FIG. 17B is a perspective view of the embodiment of FIG. 17. FIG. 17C is an end view of the seal showing the central bore in the seal design.

FIG. 18 is cross-sectional view of the plunger, seal and tubular barrel showing the seal forces on the oximetry probe produced by the seal; depression of the plunger causes the tip of the plunger to compress and deform the seal and thereby reduce the radial inward forces on the probe, allowing the probe to be advanced through the slit in the seal and through the fluid seal assembly.

FIG. 19 is a cross-sectional view of the tubular stop member of FIG. 1, showing the stop that limits the distance the sheath of the oximetry probe may be advanced through the tube towards the central venous catheter. FIG. 19A is a sectional view of the stop member along the lines 19A-19A of FIG. 19.

### DETAILED DESCRIPTION OF PREFERRED AND ALTERNATIVE EMBODIMENTS

Overview and Oximetry Probe Assembly

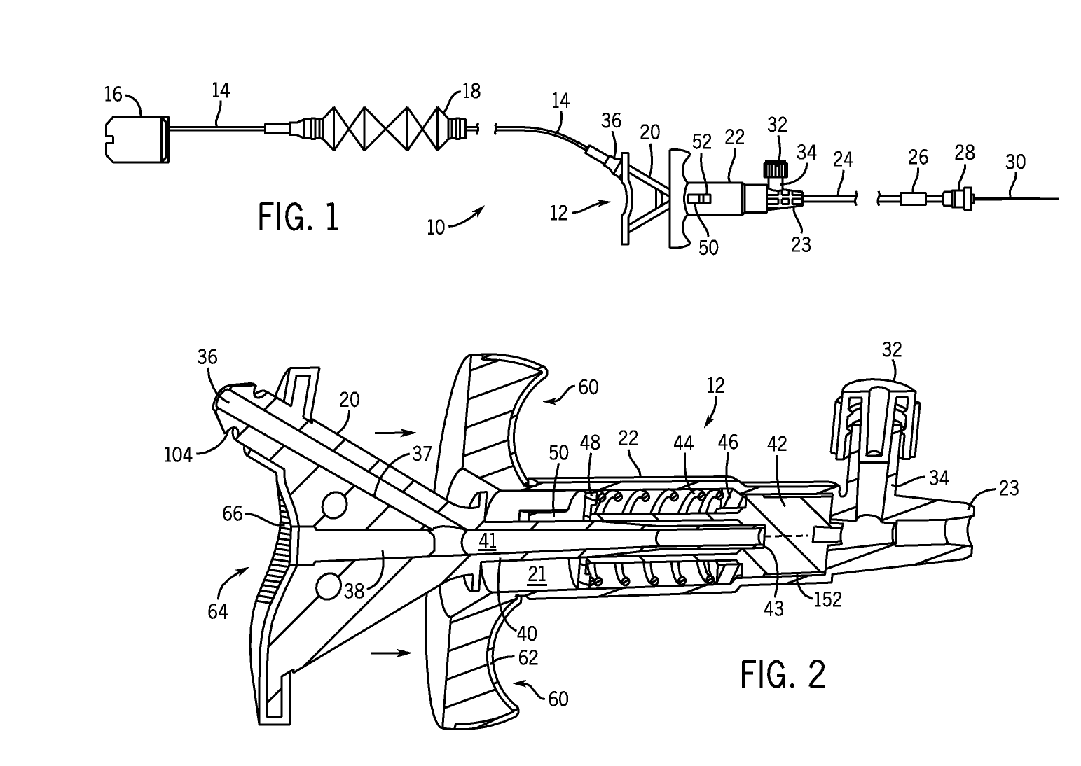

Referring now to FIGS. 1 and 2, an oximetry probe assembly 10 is shown. Assembly 10 is suitable for use, for example, in conjunction with a central venous catheter (not shown) for measurement of oxygen saturation of venous blood of a human or animal patient. The assembly 10 includes a fluid seal assembly 12 which receives an oximetry probe 14. The oximetry probe 14 includes optical fibers (not shown) that are conventional and known in the art, encased in a polymer sheath. For example, the optical fibers can be positioned within a single polyurethane sheath. If desired, the rigidity of the optical fibers and the sheath disposed about the optical fibers can be increased by adding fibers and/or wires into the optical fiber assembly. In order to provide radiopacity to the probe 14, a tantalum or other radiopaque marker can be included in the optical fiber assembly and/or probe 14 generally. The distal tips of the optical fibers can be covered with epoxy in order to protect them from damage as they are inserted into the vasculature of a patient.

The probe 14 has a proximal end portion in which the optical fibers are electrically coupled to an optical connector 16 designed to be connected to a light generator/detector system configured for determining blood oxygenation levels based upon the afferent and efferent light transmitted through the optical fibers using algorithms and hardware that is conventional in the art.

A plastic protective sleeve 18 receives the oximetry probe and has a connector at the distal portion thereof that is coupled to a barb fitting 104 on the inlet port 36 of the fluid seal assembly. Sleeve 18 is designed to maintain the sterility of the sheath of the oximetry probe 14.

The fluid seal assembly 12 includes a plunger 20 coupled to and movable relative to a barrel-shaped member 22. It will be appreciated that member 22 can have a variety of shapes and sizes without departing from the scope of the present invention.

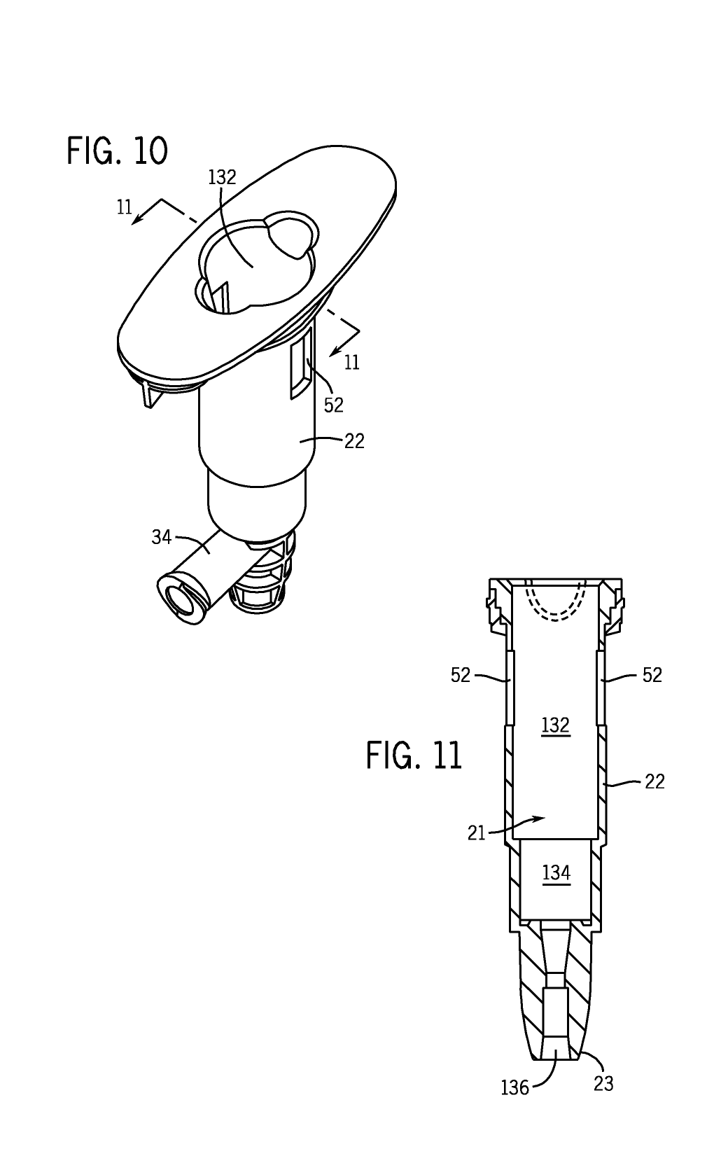

Seal 42 (FIG. 2) is positioned in the interior of the barrel-shaped member 22 and forms a fluid-tight seal to prevent flow through member 22. Seal 42 defines a slit therethrough of size and configuration sufficient to receive oximetry probe 14 therethrough. Seal 42 is configured such that it provides a fluid seal about the peripheral surface of oximetry probe 14 when probe 14 is positioned through seal 42, thereby fluidly sealing member 22. The details of the construction and operation of the fluid seal assembly 12 will be explained in detail below in conjunction with FIGS. 2-18.

Barrel-shaped member 22 includes an outlet port 23 through which the oximetry probe passes. A tubular member 24 has a first end thereof connected to the outlet port 23 and a second end thereof connected to a tubular stop member 26. Tubular member 24 can be connected to barrel-shaped member 22 using a variety of known techniques, including mechanical connections or through the use of bonding materials such as solvent bonds. Tubular member 24 can also be unitarily formed with barrel-shaped member 22.

Stop member 26 limits the insertion distance of the oximetry probe 14 into the tube 24, thereby correctly positioning the tip of oximetry probe 14 relative to the central venous catheter connected to connector 28. Connector 28 can have a variety of known constructions, e.g., a luer or locking luer construction, for operably connecting tube 24 to a central venous catheter. The distal end of the oximetry probe 14 includes a portion 30 that is configured to extend beyond the distal end of the sheath of the central venous catheter a predetermined amount when oximetry probe 14 is operably positioned relative to assembly 12. The portion 30 includes afferent and efferent optical fibers and may also include a central guide wire lumen. The stop member 26 is constructed to prevent further insertion of oximetry probe 14 into assembly 12 beyond the point at which portion 30 is properly positioned relative to the central venous catheter connected to assembly 12 using connector 28. That is, stop member 26 limits the insertion distance of the oximetry probe 14 into the tube 24.

FIG. 19 shows the tube 24, the stop member 26 and the oximetry probe sheath 14A and central guide wire and optical fibers 14B. The stop member 26 includes a tubular body having a central channel 27 for receiving distal end 14C of sheath 14A. The tubular body of stop member 26 has a tapered opening forming a first tubular portion 302 having an inside diameter greater than the outside diameter of sheath 14A and a second tubular portion 304 having an internal diameter less than the outside diameter of sheath 14A so as to prevent insertion of the sheath into the second portion 304. When the distal end 14C of the sheath 14A is inserted into the stop member 26 it abuts a stop 300 at the transition between the first portion 302 and the second portion 304, thereby preventing further insertion of the probe. The optical fibers and guide wire 30 extend beyond the stop member 26 a predetermined distance when sheath 14A abuts stop 300.

Central channel 27 can be constructed such that it permits fluids to flow around the perimeter of sheath 14A when sheath 14A is positioned therein. In the embodiment depicted in FIG. 19A, the walls of the stop member in the vicinity of the stop 300 have a triangular shape whereby the peripheral portions 306 of the triangular shape allow fluid to flow around the periphery of the tip of the sheath 14A. It will be appreciated that a variety of configurations can be used in order to provide for fluid flow about the periphery of sheath 14A.

The barrel-shaped member 22 further includes a port 34 having a cap 32. During use, the cap 32 can be removed to provide fluid access to the interior of tube 24. This enables an operator to introduce or withdraw fluids from tube 24 during use. For example, a source of biocompatible fluid such as saline solution can be connected to the port 34 so as to permit the introduction of flushing fluids into the tube 24 downstream of the seal 42.

With reference to FIGS. 1 and 2, the overall operation of the fluid seal assembly 12 will now be described. FIG. 2 shows the fluid seal assembly 12 in cross-section, but without the oximetry probe inserted therein. As noted above, the fluid seal assembly 12 includes a plunger 20 member having an inlet port 36 and a central channel 37 for receiving the oximetry probe 14. Plunger member 20 is movable relative to the barrel-shaped member 22. In the depicted embodiment of the present invention, biasing means 44 is positioned within barrel-shaped member 22. Biasing means may take the form of a compression spring that biases the plunger 20 to an extended (inactivated) position away from barrel-shaped member 22. When plunger 20 is in its inactivated position, seal 42 is not deformed. Barrel-shaped member 22 includes an outlet port 23.

Oximetry probe 14 is adapted for insertion through fluid seal assembly 12 from the inlet port 36, through the channel 37, and through outlet port 23. As above-discussed, fluid seal assembly 12 further includes a resilient, deformable seal 42 that fluidly seals the interior of barrel-shaped member 22. Seal 42 also forms a fluid seal about the periphery of oximetry probe 14 when oximetry probe 14 is inserted through the fluid seal assembly 12 and through seal 42. Plunger 20 is movable against the force of the biasing spring 44 between in inactivated position and an activated position. During movement of plunger 20 from its inactivated position to its activated position, tip 43 of plunger 20 acts on the seal 42 to deform the seal 42. Deformation of seal 42 causes the slit defined through seal 42 to enlarge, thereby facilitating insertion of oximetry probe 14 through seal 42. It will be appreciated that oximetry probe 14 is preferably relatively flexible, thereby ensuring that it does not injury the vasculature of a patient into which it is introduced. Because of the flexibility of oximetry probe 14, and because seal 42 is constructed to provide a fluid seal about the periphery of oximetry probe 14 when plunger 20 is in its inactivated position, it is necessary to “open” the slit defined through seal 42 in order to push/insert oximetry probe 14 through seal 42. However, it will be appreciated that seal 42 merely fluidly seal against the peripheral surface of oximetry probe 14 rather than clamping or locking onto oximetry probe 14. Thus, oximetry probe 14 can be pulled through the slit defied through seal 42 when plunger 42 is in its inactivated position. In short, oximetry probe 14 is somewhat like a wet noodle, i.e., it is easy to pull a wet noodle, but quite difficult to push a wet noodle.

In the illustrated embodiment, barrel-shaped member 22 includes finger grips 60 for receiving the fingers or an operator during use. The plunger includes a handle portion 66 for placement of the thumb during use. The finger grips 60 on the barrel and the handle portion 66 of the plunger form a one-handed grasping assembly, whereby a user may grasp the plunger 66 and finger grips 60 with one hand and depress the plunger 20 with the thumb relative to the barrel 22. This action actuates the seal 42. The seal 42 includes an axial slit, or alternatively a bore. The actuation of the seal 42 by the tip 43 of the plunger 42 deforms the seal 42 and permits the oximetry probe 14 to be advanced through the seal. While the user is depressing the plunger 22, they may manually advance the oximetry probe (with their other hand) through the fluid seal assembly 12 until the distal end of the oximetry probe 14 contacts the stop member 26 of FIG. 1, thereby properly positioning the oximetry probe. The user then releases the plunger 20. The spring 44 biases the plunger 20 to its extended position. The seal 42 is then compressed against the walls of the barrel 22 and the shield of the probe passing through the seal slit to form a fluid-tight seal about the oximetry probe. Fluid Seal Assembly 12

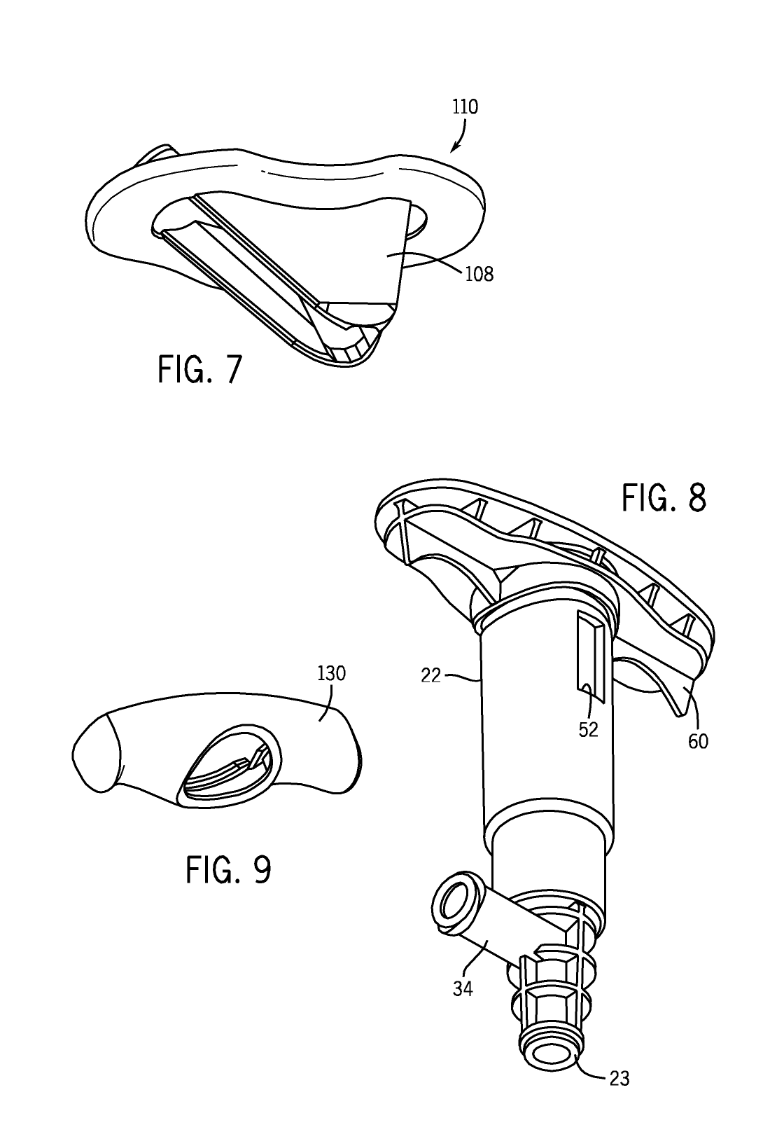

With the above discussion in mind, the construction and operation of the fluid seal assembly 12 will now be described in further detail with reference to FIGS. 2-18, with particular reference to FIGS. 2, 12 and 14. The plunger 20 includes the opening or entrance port 36 for receiving the oximetry probe 14 and a body 108 defining a channel 37. The plunger 20 includes a central bore 41 defined by a cylindrical portion 40 through which the oximetry probe is inserted. The tip 43 of the plunger acts on the seal 42 when the plunger is depressed relative to the barrel 20. A plug 38 (FIG. 2) is provided for obstructing the proximal portion of the central bore 41. The plunger includes a handle portion 66. A soft rubber or rubber-like cap 64 is placed over the handle portion 22 as shown in FIGS. 2 and 5 to aid in manual gripping of the plunger. The rubber or rubber-like cap can take other forms as described in FIGS. 6 and 7.

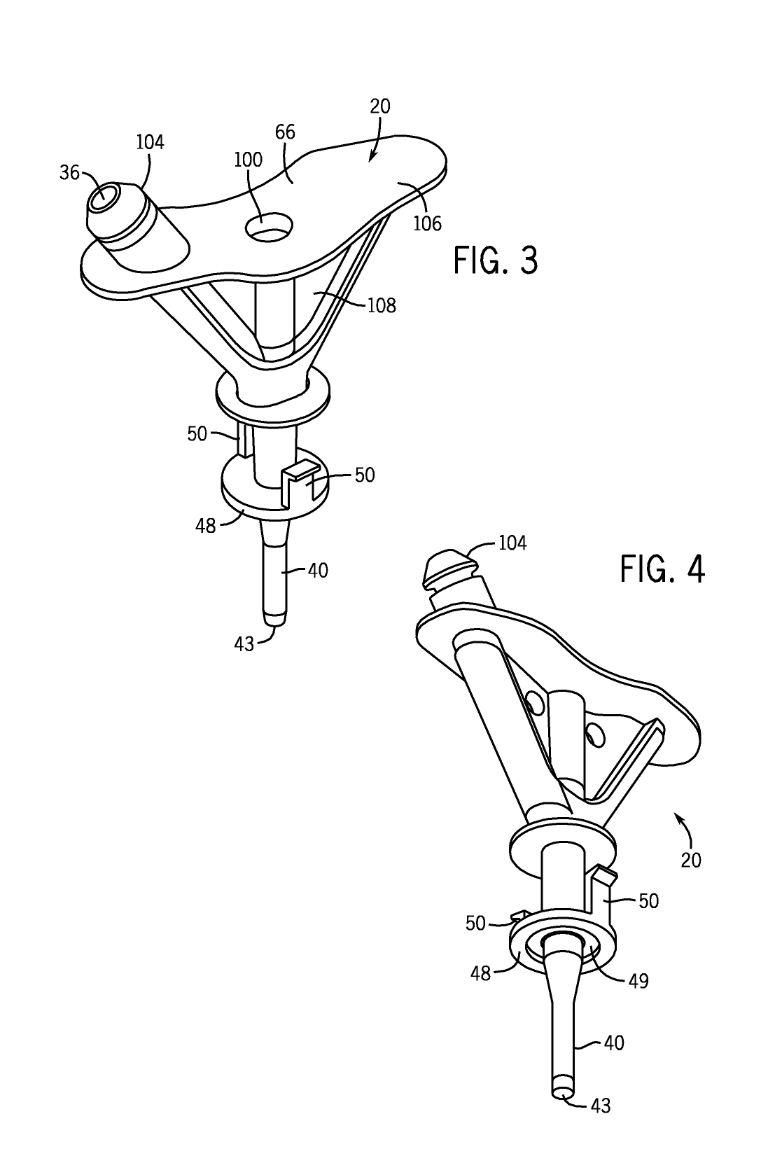

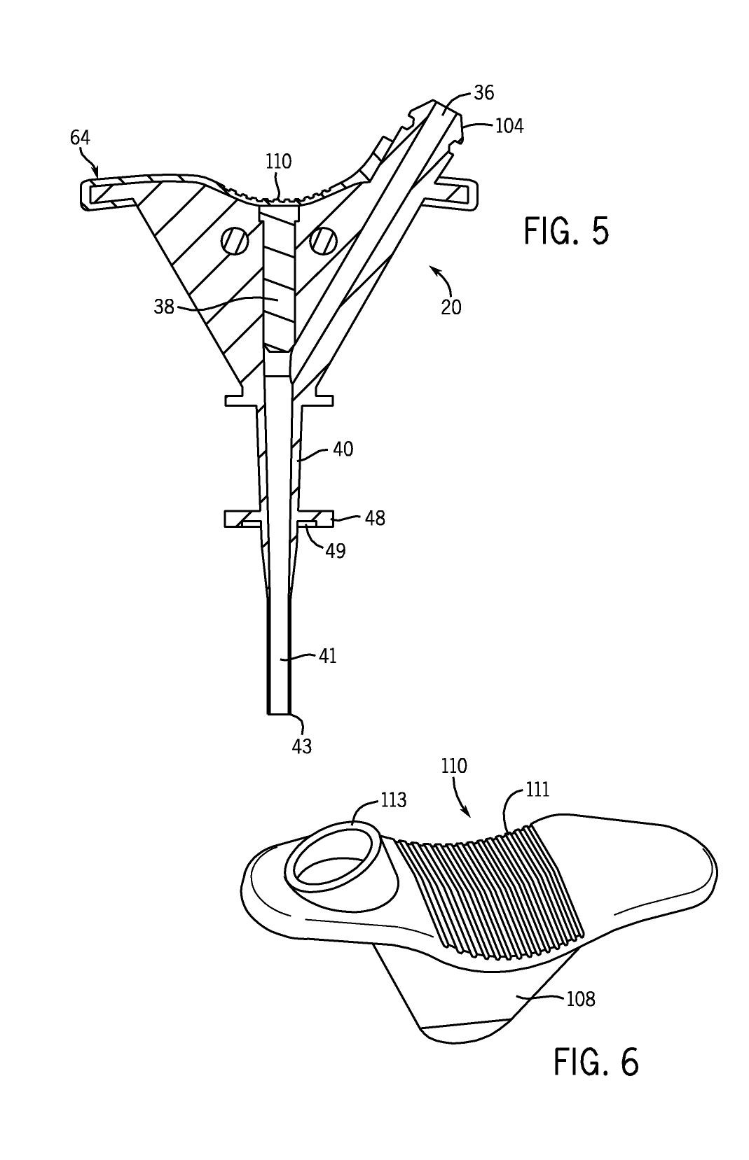

The barrel 22 includes finger grips 60. A soft rubber or rubber-like cap 62 is placed over the finger grips to aid in gripping of the barrel 22 with the index and middle fingers during use. The barrel includes a tubular body for receiving the seal 42. A spring seat 46 is placed inside the central channel 21 (FIG. 2) of the barrel 22. The lower end of the coil spring 44 seats on spring seat 46 and the upper end of the coil spring 44 seats against a circular flange 48 projecting outward from the cylindrical portion 40. The cylindrical portion 40 includes a pair of tabs 50 (FIGS. 3, 4) extending from the flange 48 which project through a pair of windows 52 (FIGS. 8, 10, 1) on opposite sides of the barrel 22 to thereby couple the plunger 20 to the barrel 22 and prevent rotation of the plunger relative to the barrel. The axial length of the windows 52 is such that it permits the tabs (and attached plunger) to move an axial distance relative to the barrel 22 so as to permit the tip 43 of the plunger to actuate the seal 42 and permit the distal end of the oximetry probe to be advanced through the seal in the manner described herein.

With reference to FIGS. 3 and 4, the plunger 20 includes a portion 106 defining the top of the plunger, the central part having a contour thereof forming the surface 66 (“handle”) which receives the thumb during use. The opening 100 is closed off with the plug 38 (FIG. 2). A barb fitting 104 is provided for receiving the distal end of the protective sheath 18 of FIG. 1. As shown in FIG. 4, the underneath surface of the flange 48 acts as a seat for the spring 44. The recessed portion 49 (FIG. 3) receives the end of the cylindrical portion of the seal 42 as shown in FIG. 2.

The plunger 20 and barrel 22 can be made using any convenient manufacturing technique such as plastic injection molding.

FIGS. 6 and 7 illustrate an alternative construction to the grip which covers the plunger 20. The grip 110 includes a knurled gripping surface 111 and an opening 113 for fitting over the barb 104 (FIG. 3). Side walls 108 cover the body portion 108 (FIG. 3) of the plunger.

The barrel 22 will now be described further with reference to FIGS. 1, 2 and 8-14. The barrel 22 includes a tubular body with integral finger grips 60, flushing port 34 and exit port 23. The body of the barrel 22 includes a central channel 21 (FIG. 1) having an upper portion 132 receiving the plunger and a lower portion 134 for receiving the seal 42. The barrel further includes an exit channel 136 which receives the oximetry probe when it has been inserted past the seal 42. The tube 24 (FIG. 1) connecting the fluid seal assembly 12 and the stop member 36 is bonded over the exit port 23.

A soft rubber or rubber-like gripping cap 130 (FIG. 9) may be fitted over the finger grips 60 to more readily facilitate gripping of the barrel with the index and middle fingers during use.

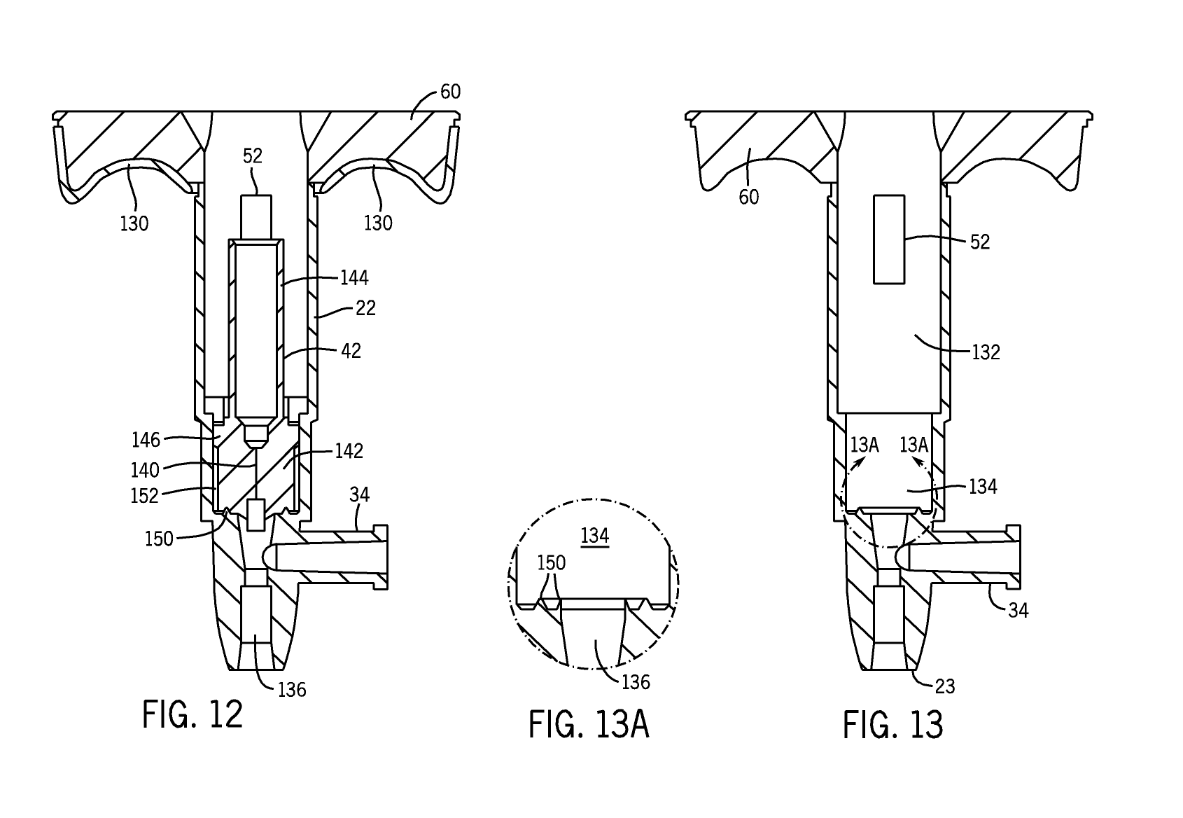

As shown in FIGS. 12 and 13, the base of the channel 134 includes a seat for the resilient, elastomeric seal 42. The seat includes projections 150 which project into the seal body to stabilize the seal during actuation by the plunger tip during use.

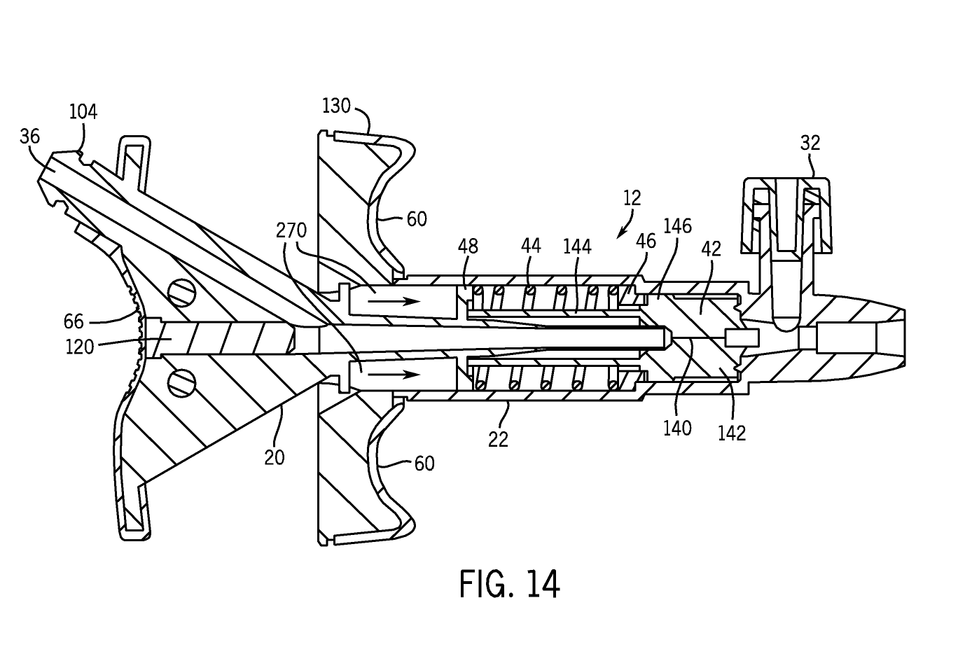

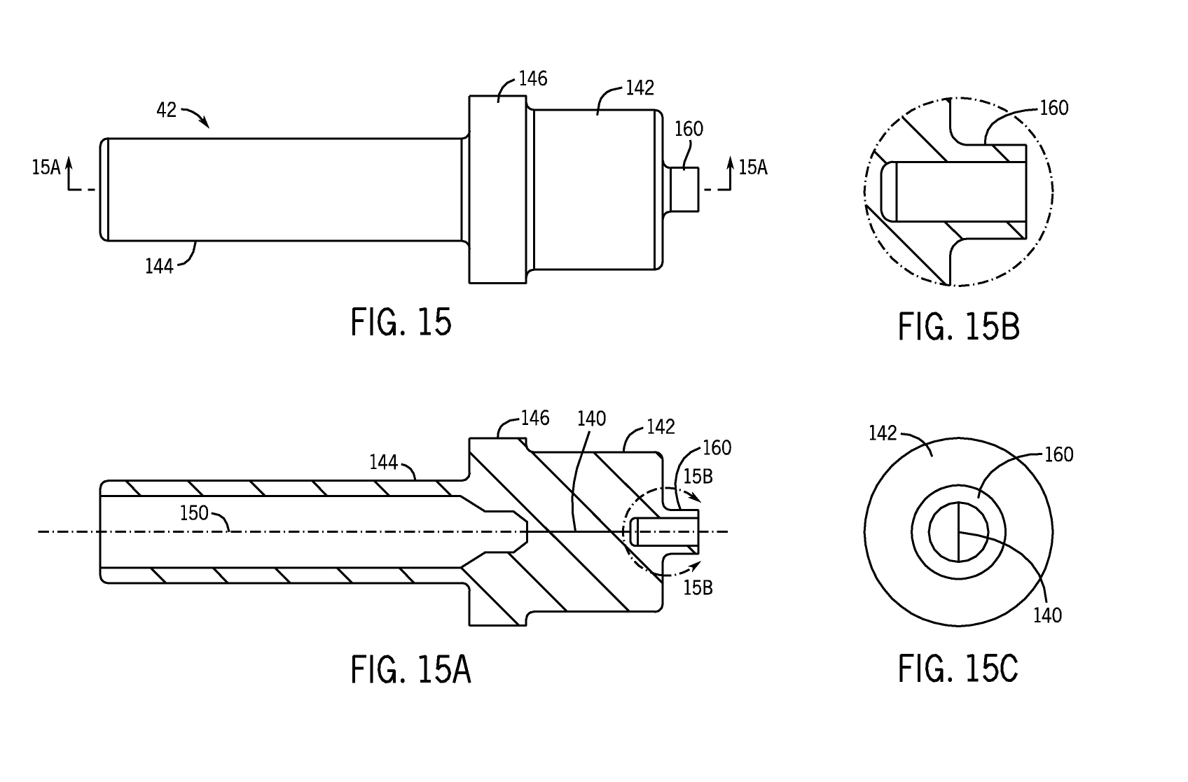

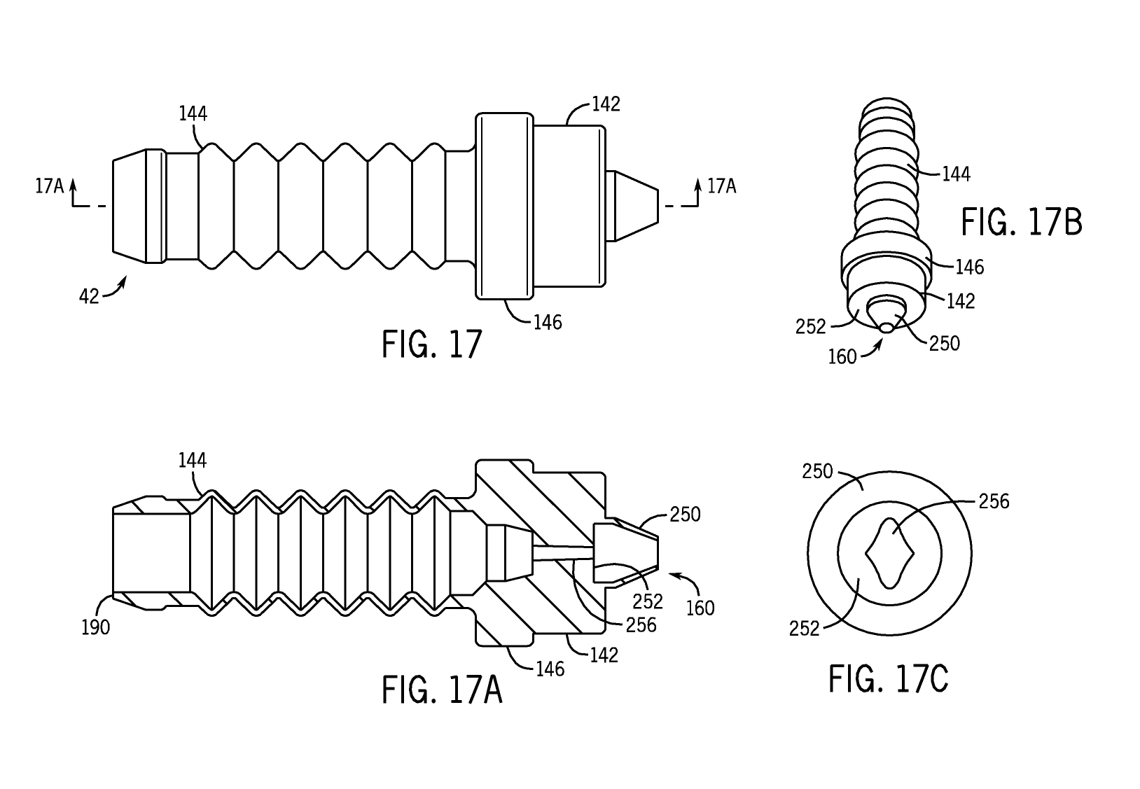

With reference to FIGS. 2, 12, 14 and 15, the seal 42 includes an elongate cylindrical portion 144 in a “stovepipe” configuration defining a longitudinal axis 150, and a solid body portion 142 integral with the elongate cylindrical portion 144. An opening feature is formed in the solid body portion 142 in substantial alignment with the axis 150. The opening feature permits the oximetry probe to be inserted through the solid body portion of the seal 42. The opening feature is shown in FIGS. 14 and 15A as a slit 140. In an alternative embodiment, the opening feature may take the form of a channel (see FIG. 17A and the subsequent discussion).

The body portion 142 of the seal 42 is dimensioned such that a slight gap 152 (FIG. 2) is formed between the exterior of the seal 42 and the barrel 20 side walls forming the channel 134. This gap permits the seal to deform and expand by actuation of the plunger tip 43 to as to permit the tip of the oximetry probe sheath to be inserted through the slit 140. In particular, movement of the plunger 20 towards the barrel 22 causes the flange 48 (FIG. 2) to partially collapse the cylindrical portion 144 of the seal while the tip 43 of the plunger compresses the body 142 of the seal causing the body 142 to deform by moving outwardly to take up the space in the gap 152 (FIG. 2), thereby opening, at least partially, the slit 140. This action makes it possible to manually insert the oximetry probe through the slit. The insertion is facilitated by slightly tapering the tip of the sheath of the oximetry probe 14. While the plunger remains depressed, the user continues to advance the oximetry probe through the tube 24 (FIG. 1) until the tip of the sheath seats in the stop in the stop member 26 as described previously. The user then releases the plunger 20. The biasing spring 44 moves the plunger 20 to an extended position away from the barrel 22 allowing the seal to resumes its normal shape. Radially-inward seal forces are imparted by the seal 42 on the sheath forming a fluid-tight seal about the oximetry probe.

The seal 42 includes a portion 146 which is dimensioned slightly larger than the internal diameter of the channel 134 (FIG. 13) thereby forming an additional seal.

The seal 42 further includes a lip seal 160 (FIG. 15) extending in the exit channel 136 (FIG. 12). The lip seal 160 is aligned with the longitudinal axis 150 of the seal 42. The lip seal 160 has an internal diameter which is less than the diameter of the sheath of the oximetry probe 14. Therefore, when the probe 14 has been inserted through the seal past the lip seal 160, the lip seal 160 maintains a seal over the sheath of the probe.

The seal 42 is preferably made from an elastomeric material which is soft enough to be deformed to allow opening of the slit 140 without requiring excessive force by the user and yet forms an adequate seal over the sheath. A presently preferred material is silicone rubber having a hardness of 39±5 Shore A.

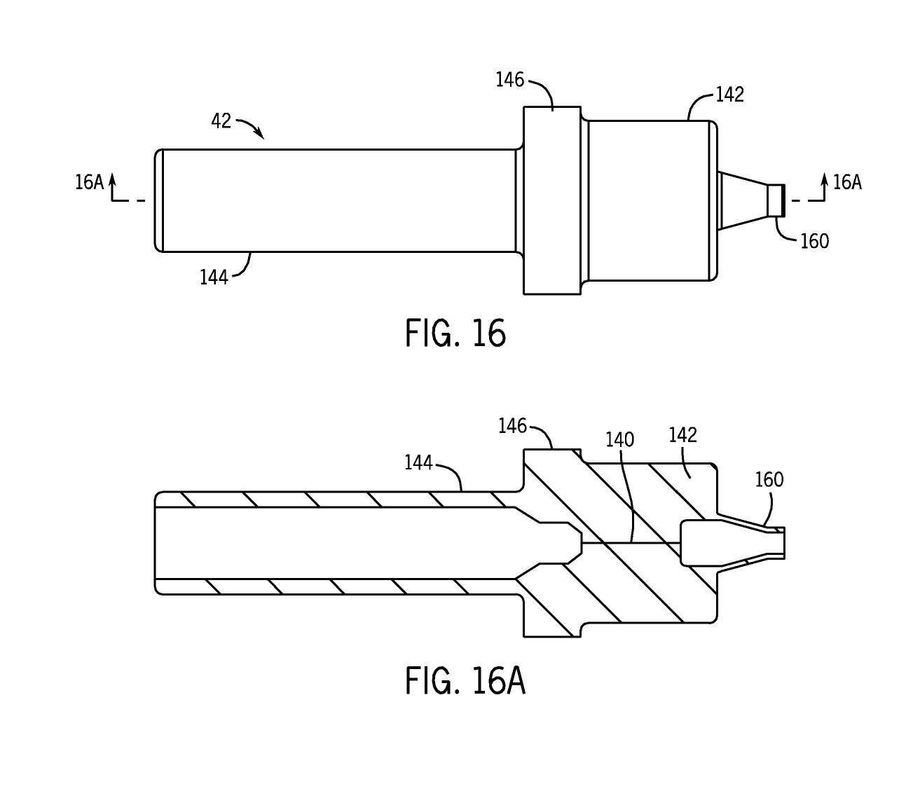

FIGS. 16 and 16A shows an alternative configuration of the seal, the main difference between the embodiment of FIG. 16 and the embodiment of FIG. 15 is the design of the lip seal 160. The embodiment of FIG. 16 has the lip seal having a tapered configuration and extends a greater distance from the face of the body portion 142.

FIGS. 17-17C show a further variation on the design of the seal 42. The cylindrical portion 144 includes a bellows configuration. The body 142 has a central bore 256 for receiving the sheath instead of a slit as in FIGS. 15 and 16. The lip seal 160 comprises a conical wall 250 extending from the end face 252 of the seal body 142. The bore 256 could be circular in cross-section, but in the example of FIG. 17C the bore has a diamond configuration.

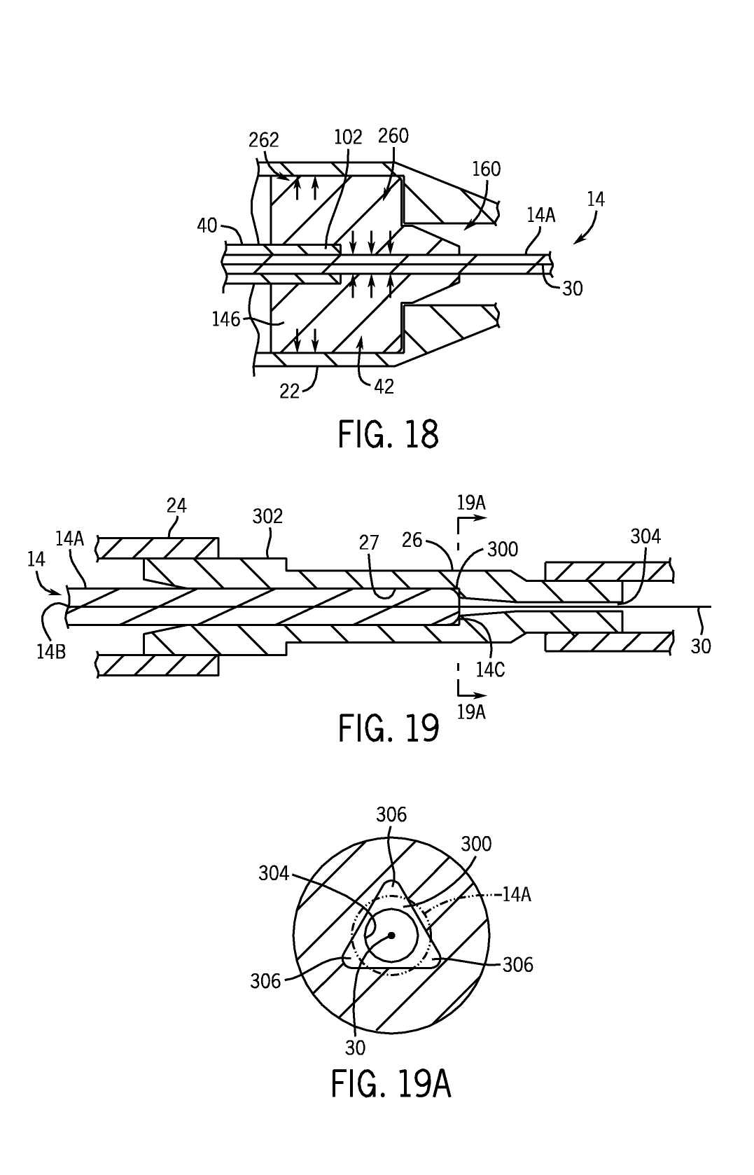

The action of the seal 42 sealing the sheath is best shown in FIG. 18. The presence of the sheath 14 within the opening of the seal (slit), coupled with the resilient characteristics of the seal 42, causes radial sealing forces shown by arrows 260 to be applied to the oximetry probe sheath 14A. Additionally, the portion 146 of the seal exerts radially outward forces on the walls of the barrel defining the channel 134, indicated by arrows 262. The lip seal 160 seals around the periphery of the sheath 14A as shown in FIG. 18.

As shown in FIG. 2, the biasing spring 44 biases the plunger 20 to its extended position such that the plunger is not actuating the seal 42. In one variation, the biasing of the plunger could be provided by a portion of the seal 42 itself. For example, in the embodiment of FIG. 17, the bellows configuration 144 is such that when the tip 190 of the bellows is seated within the pocket 49 of the flange 48 of the plunger (see FIGS. 2, 4), when the plunger 20 is depressed towards the barrel 22 the bellows collapses partially to allow the seal to be opened in the manner described herein, but the bellows also exerts forces counteracting the force on the plunger provided by the user's thumb. When the thumb is released from the plunger 20, the bellows 144 expands back to its original configuration and thus acts as a spring to move the plunger 20 to its extended position.

As will be appreciated by FIGS. 1, 2 and 15, the finger grips of the barrel 22 and the handle 66 of the plunger 20 are such that the combined plunger and barrel assembly form a one-handed grasping assembly whereby a user may grasp the plunger and finger grips with one hand (index and middle fingers under the finger grips 60 and thumb placed over the handle 66). The user depresses the plunger 66/20 with their thumb to move the plunger relative to the barrel 22 (indicated by arrows 270 of FIG. 14). This action causes the seal to be actuated by the tip 43 and flange 48 of the plunger acting on the seal 42 in the manner described herein. The deformation of the seal allows the oximetry probe 14 to be advanced through the slit through the fluid seal assembly 20.

Method of Use

The use of the assembly 10 of FIG. 1 will now be described primarily by reference to FIGS. 1 and 14. The method allows an oximetry probe 14 having a distal end 30 to be advanced through a fluid seal assembly 12. The method may be described as follows. As a preliminary matter, the luer connector 28 is connected to a central venous catheter and the optical connector 16 is plugged into the blood oximetry instrument. The user grasps the fluid seal assembly 12 with one hand by placing the index and middle fingers under the finger grips 60 and the thumb over the plunger 20. With their other hand, the user inserts the distal end 30 of the oximetry probe 14 into the fluid seal assembly 12 by inserting the end 30 into the inlet port 36 on the plunger. The user then further inserts the oximetry probe 14 through the fluid seal assembly 12 until the distal end 30 of the oximetry probe is approximately in the vicinity of the seal 42. (In the slit embodiment, the slit is normally closed and the user will feel the tip of the probe 14 making contact with the seal 42, preventing further insertion of the probe through the assembly. In the embodiment with the seal having a central bore (FIG. 17), the fiber optics and guidewire (portion 30 of FIG. 1) may advance through the bore but the tip of the sheath 14C will abut the entrance to the bore and such action will be felt in the hands of the user.) The user then manually depresses the plunger with their thumb, moving the plunger 20 relative to the barrel 22 to thereby actuate the seal with the plunger 20. This action opens the slit in the seal and permits the distal end of the oximetry probe 14 to be advanced through the seal 42. (In the embodiment with the seal having a central bore (FIG. 17), this action permits the sheath to be advanced through the bore.) While the plunger is depressed, the user continues to advance the oximetry probe with their other hand through the fluid seal assembly 12 and through the outlet port 23 of the barrel 22. The user continues to press the plunger against the barrel and advance the probe through the assembly 12 until the distal tip of the sheath 14C contacts the stop 300 (FIG. 19) in the stop member 26. The user releases the plunger 20 and connects the protective sheath 18 to the barb fitting 104 on the plunger 20. The tip 30 of the probe (FIG. 19 extends through the connector 28 connecting the assembly 10 to a central venous catheter, thereby allowing optical measurements of blood oximetry to be made using optical fibers in the probe 14.

When the user releases the plunger, the actuation of the seal ends as the plunger 20 is moved by the spring 44 to its extended position. The seal 42 compresses against the sheath of the probe 14 as shown in FIG. 18, preventing fluids from passing around the sheath into the channels 37, 41 of the plunger 20 or the channel 21 of the barrel 22 (FIG. 2). The user may connect a source of blood-compatible solution to the port 34 by removing the cap 32 (FIG. 2) and attaching a tube to the port e.g., using a luer connector connected to the source of solution which attaches to the port 34. Fluid may be infused into the barrel 22 “downstream” of the seal 42, i.e., to the right of the seal 42 of FIG. 2, in order to prevent blood clots from forming around the oximetry probe in the exit port 23, tube 24 or stop member 26 (FIG. 1).

While a number of exemplary aspects and embodiments have been discussed above, those of skill in the art will recognize certain modifications, permutations, additions and sub-combinations thereof as being present in this disclosure. It is therefore intended that the following appended claims and claims hereafter introduced are interpreted to include all such modifications, permutations, additions and sub-combinations as are within their true spirit and scope.

We claim

1. An oximetry probe assembly, comprising:
an oximetry probe comprising a elongate tubular member;
a fluid seal assembly having an inlet port for receiving said oximetry probe, a channel and an outlet port, said fluid seal assembly adapted for insertion of said oximetry probe through said fluid seal assembly from said inlet port through said channel to said outlet port, said fluid seal assembly further comprising a resilient, deformable seal for forming a fluid seal about said oximetry probe when said oximetry probe is inserted through said fluid seal assembly;
a tubular stop member for receiving the probe; and
a tube having a first end connected to said outlet port of said fluid seal assembly and a second end connected to said stop member, said stop member adapted to limit the insertion distance of said oximetry probe into said tube;
wherein said fluid seal assembly further comprises a manually-moveable plunger acting on said seal to deform said seal so as to permit manual insertion of said oximetry probe through said fluid seal assembly and advancement of said oximetry probe through said tube towards said stop member.

2. The assembly of claim 1, wherein said oximetry probe further comprises optical fibers and has a first end connected to an optical connector body and a second end, and a sheath enclosing said optical fibers and having a distal end, wherein said second end of said oximetry probe comprises a length of optical fibers extending beyond said distal end of said sheath, said distal end of said sheath engaging said stop member to thereby limit the insertion distance of said oximetry probe into said tube.

3. The assembly of claim 2, wherein said stop member comprises a tubular body having a central channel for receiving said distal end of said sheath, said tubular body having a first portion having an inside diameter greater than the diameter of said sheath and a stop so as to prevent insertion of said sheath past said stop, said tubular body further having at least one channel feature whereby fluids may flow around said sheath when said sheath is inserted such that said distal end of said sheath is inserted up to said stop.

4. The assembly of claim 1, wherein said plunger further comprises a body having a handle portion, said inlet port of said fluid seal assembly formed in said body,
and wherein said fluid seal assembly further comprises a barrel coupled to said plunger and having said seal placed therein;
said plunger moveable relative to said barrel wherein manual actuation of said plunger causes said plunger to move relative to said barrel to thereby deform said seal.

5. The assembly of claim 4, wherein said seal comprises an elongate cylindrical portion having a longitudinal axis and a body portion integral with said elongate cylindrical portion, and wherein an opening feature is formed in said body portion in substantial alignment with said longitudinal axis for permitting said oximetry probe to be inserted therethrough.

6. The assembly of claim 5, wherein said opening feature comprises a slit.

7. The assembly of claim 5, wherein said opening feature comprises a channel.

8. The assembly of claim 4, wherein said seal further comprises a lip seal portion extending from said body portion opposite from said elongate cylindrical portion and substantially aligned with said axis, said lip seal portion having an internal diameter less than the exterior diameter of said oximetry probe.

9. The assembly of claim 4, wherein said seal is made from an elastomeric material having a hardness of 39±5 Shore A.

10. The assembly of claim 5, wherein said elongate cylindrical portion of said seal is formed in a bellows configuration.

11. The assembly of claim 4, wherein said fluid seal assembly further comprises a biasing means for biasing said plunger relative to said barrel such that said plunger is in an extended position relative to said barrel.

12. The assembly of claim 1 1, wherein said biasing means comprises a spring.

13. The assembly of claim 11, wherein said biasing means comprises a portion of said seal.

14. The assembly of claim 4, wherein said barrel further comprises finger grips, said finger grips and said handle forming a one-handed grasping assembly whereby a user may grasp said plunger and finger grips with one hand and depress said plunger relative to said barrel to thereby deform said seal and permit said oximetry probe to be advanced through said fluid seal assembly.

15. A fluid seal assembly for an oximetry probe in the form of an elongate tubular body, comprising:
a plunger comprising a body having a first portion having an inlet port for receiving said oximetry probe and a second portion forming a channel;
a barrel comprising a tubular body having an outlet port, the plunger and barrel adapted for insertion of the oximetry probe through the inlet port of the plunger and through said channel and barrel to said outlet port; and
a resilient, deformable seal placed within said barrel for forming a fluid seal about said oximetry probe when said oximetry probe is inserted through said fluid seal assembly;
wherein said plunger and barrel are adapted for manually-actuated, relative movement therebetween wherein movement of said plunger towards said barrel actuates said seal, thereby permitting manual insertion and advancement of said oximetry probe through said fluid seal assembly.

16. The fluid seal assembly of claim 15, wherein said seal comprises an elongate cylindrical portion having a longitudinal axis and a body portion integral with said elongate cylindrical portion, and wherein an opening feature is formed in said body portion in substantial alignment with said longitudinal axis for permitting said oximetry probe to be inserted therethrough.

17. The fluid seal assembly of claim 16, wherein said opening feature comprises a slit.

18. The fluid seal assembly of claim 16, wherein said opening feature comprises a channel.

19. The fluid seal assembly of claim 16, wherein said seal further comprises a lip seal portion extending from said body portion opposite from said elongate cylindrical portion and substantially aligned with said axis, said lip seal portion having an internal diameter less than the exterior diameter of said oximetry probe.

20. The fluid seal assembly of claim 15, wherein said seal is made from an elastomeric material having a hardness of 39±5 Shore A.

21. The fluid seal assembly of claim 15, wherein said fluid seal assembly further comprises a biasing means for biasing said plunger relative to said barrel such that said plunger is in an extended position relative to said tubular body.

22. The fluid seal assembly of claim 21, wherein said biasing means comprises a spring.

23. The fluid seal assembly of claim 21, wherein said biasing means comprises a portion of said seal.

24. The assembly of claim 15, wherein said barrel further comprises finger grips, said finger grips and said handle forming a one-handed grasping assembly whereby a user may grasp said plunger and finger grips with one hand and depress said plunger relative to said barrel to thereby deform said seal and permit said oximetry probe to be advanced through said fluid seal assembly.

25. The assembly of claim 15, wherein said barrel further comprises a port for receiving a source of fluid.

26. A method of advancing an oximetry probe having a distal end through a fluid seal assembly, comprising the steps of:
(a) inserting said distal end of said oximetry probe into a fluid seal assembly comprising a plunger, a barrel and a seal disposed within said barrel, said plunger having an inlet port for receiving the oximetry probe, said barrel having an outlet port;
(b) further inserting said oximetry probe through said fluid seal assembly until said distal end of oximetry probe is approximately in the vicinity of said seal;
(c) manually moving said plunger relative to said barrel to thereby actuate said seal with said plunger and thereby permit said distal end of said oximetry probe to be advanced through said seal; and
(d) while said plunger is moved relative to said barrel, further advancing said oximetry probe through said fluid seal assembly whereby said distal end of said oximetry probe may be advanced through said outlet port of said barrel.

27. The method of claim 26, wherein the barrel further comprises finger grips, said finger grips and said plunger forming a one-handed grasping assembly, wherein step (c) comprises the step of manually grasping said plunger and finger grips with one hand and depressing said plunger relative to said barrel to thereby deform and open said seal so as to permit said distal end of said oximetry probe to be advanced through said seal.

28. The method of claim 27, further comprising the steps of:
(e) manually releasing said plunger;
(f) moving said plunger axially away from said barrel to thereby remove actuation on said seal; and
(g) forming a fluid seal around said oximetry probe with said seal.
